(19) United States
(12) Patent Application Publication
(10) Pub. No.: US 2021/0161248 A1
(43) Pub. Date: Jun. 3, 2021

(54) MANUFACTURING METHOD OF CONVEX CUSHION STRUCTURE FOR WALKING UNSTEADINESS AND ORTHOPEDIC INSOLE

(71) Applicants: Dongguan University Of Technology, Dongguan City (CN); Dongguan Juming Sports Technology Co., Ltd., Dongguan City (CN)

(72) Inventors: Nan LI, Dongguan City (CN); Jin CHEN, Dongguan City (CN); Shenggui CHEN, Dongguan City (CN)

(21) Appl. No.: 16/951,587

(22) Filed: Nov. 18, 2020

(30) Foreign Application Priority Data
Dec. 2, 2019 (CN) 201911214888.8

(51) Int. Cl. A43B 13/14; A43D 1/02; A43B 17/00; A61F 5/14

(52) U.S. Cl. CPC A43B 13/145; A43D 1/02; A43D 2200/60; A61F 5/14; A43B 17/00

(57) ABSTRACT

An orthopedic insole and a method of manufacturing a convex cushion structure for walking unsteadiness, the convex cushion structure being provided on an upper surface of an insole body. The method includes steps of data collection: measuring plantar static pressure data and plantar dynamic pressure data; data analysis: analyzing the plantar static pressure data and the plantar dynamic pressure data; preparing an insole body; performing insole modeling based on the plantar static pressure data; and importing the plantar dynamic pressure data to manufacture the insole body; partitioning the insole body; preparing the convex cushion structure: determining a shape of the convex cushion structure by using a test result of the dynamic pressure distributions; determining a specific location of the convex cushion structure by using a test result of the gait lines and the gait cycle; and printing the convex cushion structure on the upper surface of the insole body.

### CROSS REFERENCE TO RELATED APPLICATIONS

The present application claims priority to Chinese Patent Application No. 201911214888.8 (filed on Dec. 2, 2019), the entire content of which is incorporated herein by reference in its complete entirety.

### TECHNICAL FIELD

The disclosure relates to the technical field of orthopedic insoles, in particular, to a manufacturing method of a convex cushion structure for walking unsteadiness and an orthopedic insole.

### BACKGROUND

In daily life, ordinary people often suffer from walking unsteadiness. Generally, ankle unsteadiness caused by frequent sprains, heel pain and discomfort with the forefoot due to longtime standing, and abrasion of metatarsal bones will cause walking unsteadiness, which can lead to uneven force on the arch of the foot. A long-term walking unsteadiness without treatment can lead to abnormal walking posture, and can also affect hip, knees and other body parts in a serious case, which will seriously affect people's life, work and study. At present, wearing orthopedic insoles has been proven to be one of the lowest-risk and scientifically effective treatments for foot symptoms.

At present, following methods are mainly adopted for manufacturing orthopedic insoles: one method is a traditional method for manufacturing insoles using plaster model, which manufactures insoles successively by using a plaster bandage on a patient's foot to take female mold, shaping a plaster male mold, and using plastic plates for high-temperature molding or hand knocking metal materials to make the insoles. This method takes a long time to produce insoles and requires the producer has a higher technical level. Another method for manufacturing orthopedic insoles is based on patient information obtained through computers, and commonly manufactures insoles based on foot pressure; wherein a size of a flat insole is obtained by collecting foot static pressure data and generating a pressure map, a height of the insole is obtained by using formulas based on the principle of pressure dispersion to perform finite element data analysis on the foot pressure data; an insole model is obtained based on the size of the flat insole and the height of the insole; and the insole is 3D printed out. However, this method only considers the patient's static pressure without considering the tester's sports biomechanics. Therefore, the insole manufactured by this method does not meet the patient's sports biomechanics and a longtime walking wearing the insole will cause abnormal gait lines and postures, and affect hip, knee and other body parts in a serious case.

In view of this, it is necessary to provide a manufacturing method of a convex cushion structure for walking unsteadiness and an orthopedic insole to solve the existing problems.

### SUMMARY

One object of the disclosure is to provide a manufacturing method of a convex cushion structure for walking unsteadiness, which uses plantar static pressure data and plantar dynamic pressure data to make a convex cushion structure, has an advantage of maximally conforming to personal sports biomechanics. Owing to the convex cushion structure being arranged on an upper surface of the insole body, the convex cushion structure can effectively protect a patient who walks unsteadily from uncomfortable walking, with simple preparation and low cost.

Another object of the disclosure is to provide an orthopedic insole, comprising an insole body and a convex cushion structure provided on an upper surface of the insole body; wherein the convex cushion structure is manufactured by the manufacturing method of the convex cushion structure for walking unsteadiness. The orthopedic insole with simple structure and low cost can effectively protect a patient who walks unsteadily, and scientifically and effectively treats the feet of the patient who walks unsteadily.

In order to achieve the above objects, the disclosure provide a manufacturing method of a convex cushion structure for walking unsteadiness, wherein the convex cushion structure is provided on an upper surface of an insole body, and the manufacturing method comprises steps of:

(1) data collection: measuring foot pressure data of a tester under two natural states of standing and walking by using a foot pressure plate; wherein the foot pressure data comprise plantar static pressure data and plantar dynamic pressure data, and the plantar dynamic pressure data comprise dynamic pressure distributions, gait lines and a gait cycle;

(2) data analysis: determining whether pressure distributions of a left and a right feet are symmetrical, whether pressures on forefoot and hindfoot are too concentrated, whether a maximum force bearing point is moved forward, and whether there is toed-in or toed-out, according to the plantar static pressure data; and determining whether the gait lines are normal and a swing situation of the gait cycle according to the plantar dynamic pressure data; wherein, the dynamic pressure distributions are used to determine whether the pressure distributions of the left and right feet are symmetrical, whether the pressures on the forefoot and hindfoot are too concentrated, whether the maximum force bearing point is moved forward, and whether there is toed-in and or toed-out; the gait lines are used to determine whether there are situations of flatfoot, clawfoot, metatarsal pain, equinus heel pain and unsteadiness of a center of gravity; and the gait cycle is used to determine whether there is abnormal walking, and determine whether the center of gravity is unsteady by using the gait cycle in combination with the gait lines;

(3) preparation of the insole body: performing insole modeling by using an orthotics module database in an Easy CAD software, based on the plantar static pressure data; calculating a thickness of the insole by importing the plantar dynamic pressure data, and manufacturing the insole body by using 3D printing technology;

(4) partition of the insole body: dividing the insole body into a first toe region, a second toe region, a first metatarsal region, a second metatarsal region, a medial arch region, a heel region, a lateral arch region, a fifth metatarsal region, a fourth metatarsal region, and a third metatarsal region; and

(5) preparation of the convex cushion structure: determining a shape of the convex cushion structure by using a test result of the dynamic pressure distributions; determining a specific location where the convex cushion structure is located on the upper surface of the insole body by using a test result of the gait lines and the gait cycle while determining a curve radian of the convex cushion structure by using the gait lines; and then printing out the convex cushion structure on the upper surface of the insole body by using the 3D printing technology.

Optionally the step (1) further comprises: obtaining digital footprints by using a 2D scanner for grasping a shape of foot of the tester.

Accordingly, the disclosure provide an orthopedic insole, comprising an insole body and a convex cushion structure provided on an upper surface of the insole body; wherein the convex cushion structure is manufactured by the manufacturing method of the convex cushion structure for walking unsteadiness.

Optionally, a bottom of the insole body is provided with a plurality of honeycomb structures.

Optionally, the plurality of the honeycomb structures penetrate the insole body.

Optionally, axial heights of the plurality of honeycomb structures are different.

Optionally, the honeycomb structure is formed as a hexagon.

Optionally, a distance between opposite sides of the hexagon is 2.5 mm.

Optionally, a distribution density of the honeycomb structures in the insole body is 8 pcs/cm2.

Compared with the prior art, the orthopedic insole provided by the disclosure uses the plantar static pressure data and the plantar dynamic pressure data to design a personalized orthopedic insole, especially uses three kinds of data, comprising the dynamic pressure distributions, the gait lines and the gait cycle, to analysis to obtain accurate data, and at the same time, uses the orthopedic insoles with the honeycomb structure, different from those on the market using thickness for orthotics, to improve the orthopedic effect, prolong the service life and save raw materials.

### BRIEF DESCRIPTION OF DRAWINGS

FIG. 1 is a schematic structural diagram of the orthopedic insole provided by the disclosure with the convex cushion structure hidden.

FIG. 2 is a schematic structure diagram of the orthopedic insole provided by the disclosure.

FIG. 3 is a top view of the convex cushion structure in the orthopedic insole shown in FIG. 2.

FIG. 4 is a front view of the convex cushion structure in the orthopedic insole shown in FIG. 2.

FIG. 5 is a side view of the convex cushion structure in the orthopedic insole shown in FIG. 2.

FIG. 6 is a schematic structural diagram from another view of the orthopedic insole provided by the disclosure.

FIG. 7 is a test chart of the tester's plantar static pressure data before orthotics.

FIG. 8 is a test chart of the tester's plantar static pressure data after orthotics.

FIG. 9 is a test chart of dynamic pressure distribution data before orthotics.

FIG. 10 is a test chart of dynamic pressure distribution data after orthotics.

FIG. 11 is a test chart of gait cycle data before orthotics.

FIG. 12 is a test chart of 2D scanner measurement data.

### DETAILED DESCRIPTION

The disclosure will be further explained below in combination with specific embodiments. The description is more specific and detailed, but it should not be interpreted as a limitation to the scope of the disclosure. All technical solutions obtained by equivalent replacements or equivalent changes shall be included in the protection scope of the claims of the disclosure.

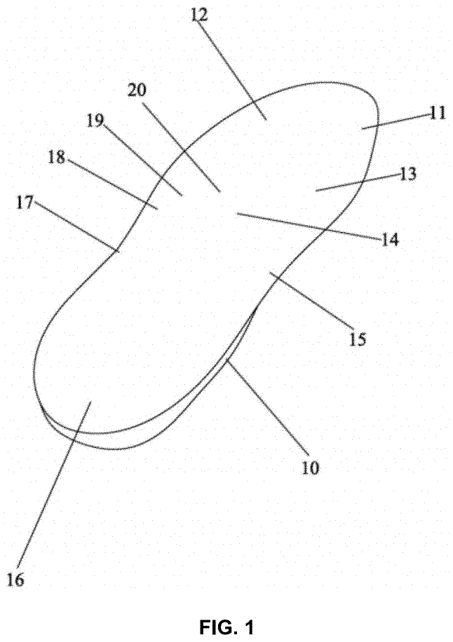

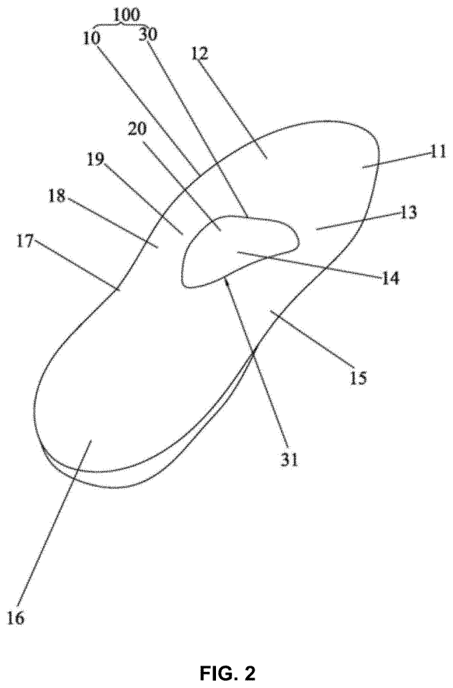

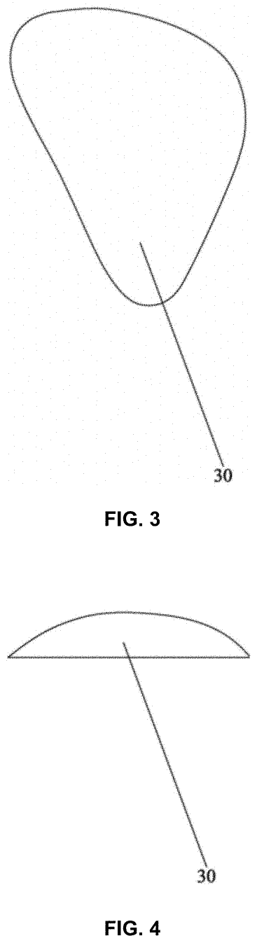

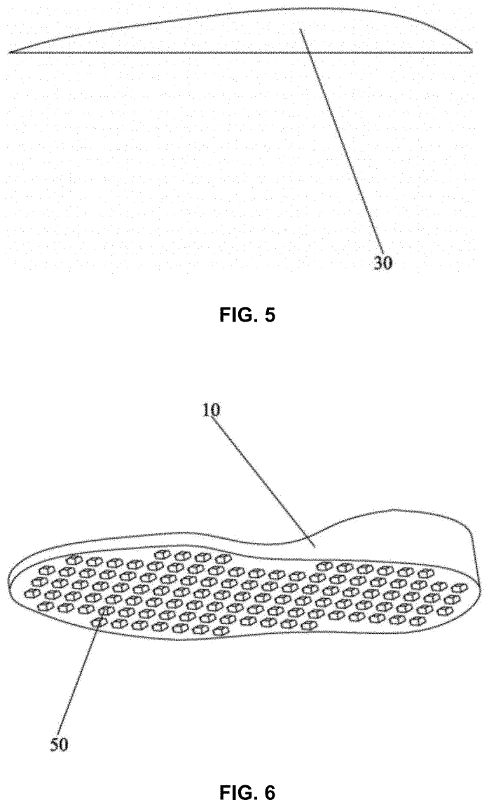

As shown in FIG. 1 and FIG. 2, the orthopedic insole 100 provided by the disclosure comprises an insole body 10 and a convex cushion structure 30 provided on the upper surface of the insole body 10. The convex cushion structure 30 is manufactured by the manufacturing method of the convex cushion structure for walking unsteadiness.

Specifically, as shown in FIG. 6, the bottom of the insole body 10 is provided with a plurality of honeycomb structures 50, resulting in less raw material waste, good comfort and lower cost. Further, axial heights of the plurality of honeycomb structures 50 can be differently designed according to actual stress distributions. The honeycomb structures 50 penetrate the insole body 10 vertically. When the honeycomb structures 50 are subjected to a load perpendicular to a plate surface, the bending rigidity of the honeycomb structures 50 is almost the same as, or even higher than, that of a solid plate having a same material and thickness with the honeycomb structures 50, but the weight of the honeycomb structures 50 is 70-90% lighter than the solid plate, and meanwhile, the honeycomb structures 50 have advantages of less deformation, crack and break, shock absorption, sound insulation, heat insulation and strong weatherability. The relevant parameters such as the pore size of the honeycomb structure can be selected according to needs, and are not limited here. In one embodiment, the honeycomb structure 50 is formed as a hexagon, especially a regular hexagon. It's found through experimental studies that, when a distance between opposite sides of the honeycomb structure 50 is 2.5 mm and a distribution density is 8 pcs/cm2, and once the size increases by 0.5 mm, the distribution density decreases by 1 pcs/cm2, better anti-vibration, anti-shake effect, and steadiness and longer service life can be achieved. In one embodiment, the distance between the opposite sides of the honeycomb structure 50 is 2.5 mm, and the distribution density is 8 pcs/cm2, but it is not limited to this. The orthopedic insole 100 with the honeycomb structures 50 is different from those on the market using thickness orthotics, which improves the orthopedic effect and prolongs the service life.

In one embodiment, the manufacturing method of a convex cushion structure for walking unsteadiness comprises following steps:

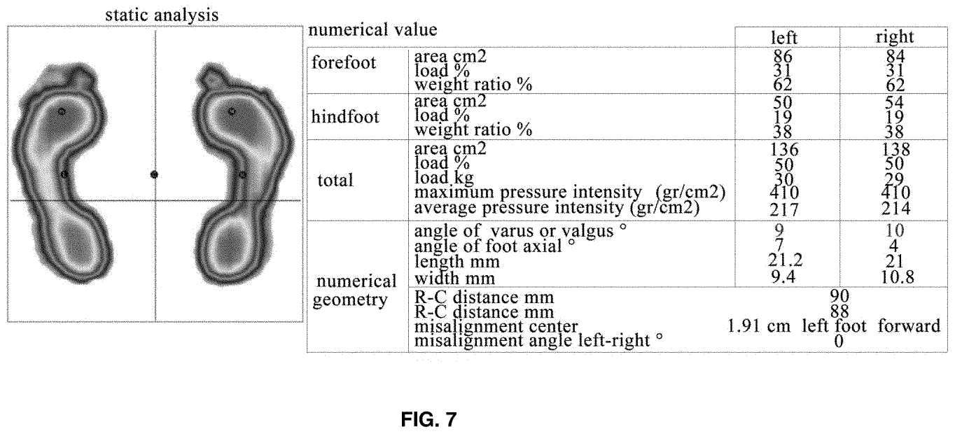

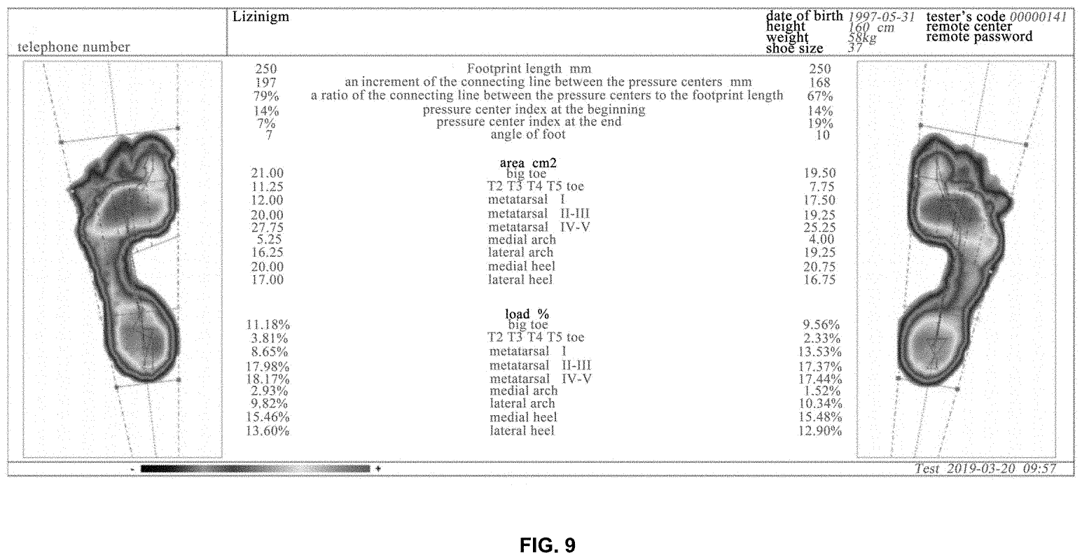

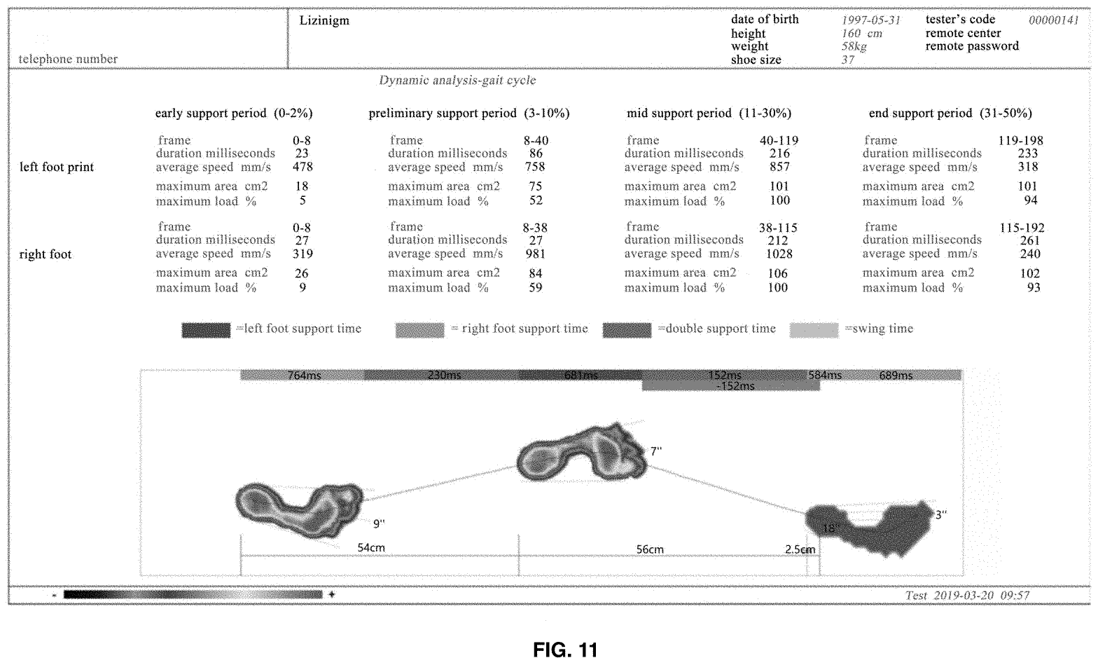

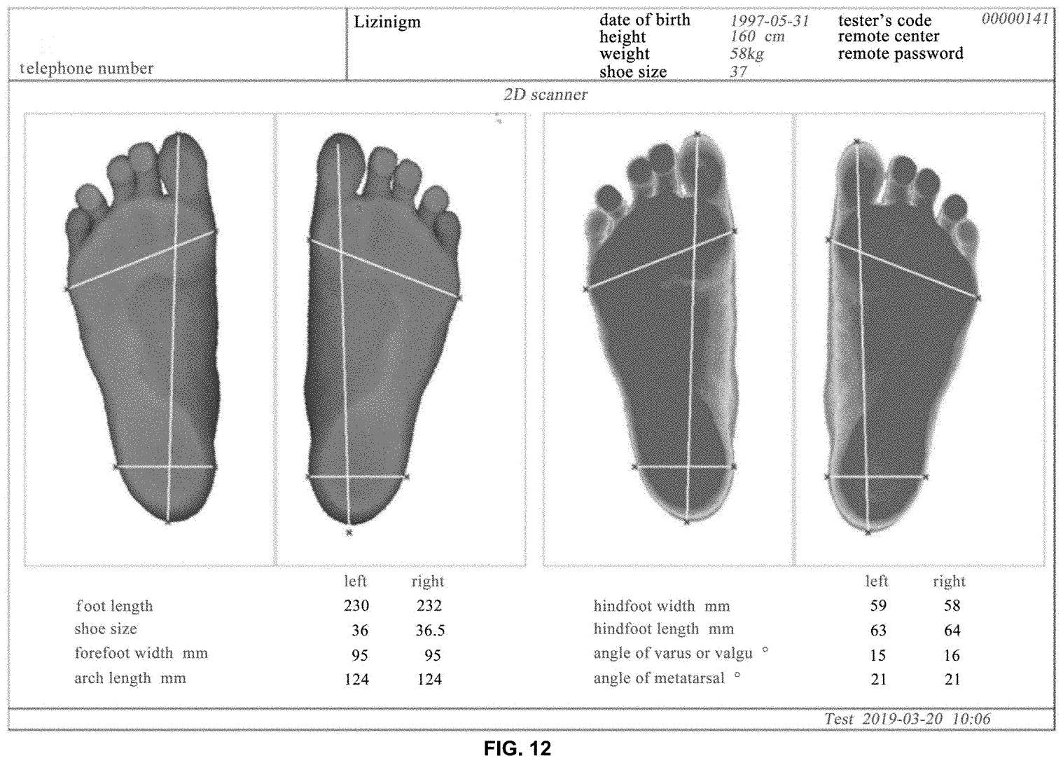

(1) Data collection: measuring foot pressure data of the tester under two natural states of standing and walking by using a foot pressure plate, which requires the tester looking forward and breathing relaxedly and naturally; wherein the foot pressure data comprise plantar static pressure data (as shown in FIG. 7) and plantar dynamic pressure data. The foot pressure plate feeds back collected plantar static pressure data and plantar dynamic pressure data to the computer controller, and the computer controller uses a software measurement system (such as FEREESTEP) to generate the dynamic pressure distributions, the gait cycle and the gait lines (as shown in FIG. 9 and FIG. 11). A 2D scanner is used to measure an angle of the hallux and an angle of the metatarsal, wherein the angle of the hallux is used to determine whether there is hallux valgus, and the angle of the metatarsal is used to determine whether there is metatarsus varus or metatarsal valgus. Please refer to the test chart of 2D scanner measurement data shown in FIG. 12, in which the angle of the metatarsal is 21, which is normal.

(2) Data analysis: determining whether pressure distributions of left and right feet are symmetrical, whether pressures on forefoot and hindfoot are too concentrated, whether a maximum force bearing point is moved forward, and whether there is toed-in or toed-out according to the plantar static pressure data; and determining whether the gait lines are normal and a swing situation of the gait cycle according to the plantar dynamic pressure data; wherein, the dynamic pressure distributions are used to determine whether the pressure distributions of the left and right feet are symmetrical, whether the pressures on the forefoot and hindfoot are too concentrated, whether the maximum force bearing point is moved forward, and whether there is toed-in or toed-out; the gait lines are used to determine whether there are situations of flatfoot, clawfoot, metatarsal pain, equinus heel pain and unsteadiness of a center of gravity; and the gait cycle is used to determine whether there is abnormal walking, and determines whether the center of gravity is unsteady in combination with the gait lines.

In one embodiment, as shown in FIG. 7, step (1) is adopted to measure the test chart of the tester's plantar static pressure data before orthotics. It can be seen from FIG. 7 that the weight ratio of the forefoot is 62%, and the weight ratio of the hindfoot is 38%, both of which are quite different from a normal value (45%-50%), thus it is determined that the pressures of the forefoot and hindfoot are too concentrated on the forefoot, and the maximum force bearing point is moved forward, resulting in walking unsteadiness.

As shown in FIG. 9, step (1) is adopted to measure the test chart of dynamic pressure distribution data before orthotics, wherein the line trends in FIG. 9 represent the gait lines. FIG. 11 is the test chart of the gait cycle data before orthotics. It can be seen from FIG. 9 and FIG. 11 that the pressure on the tester's forefoot is too high, the heel is unsteady, and the gait lines are abnormal. The normal gait line is approaching the foot type, which starts from the heel to the arch, passing through the fourth and fifth metatarsals, second and third metatarsals, and finally extending out from the big toe. In FIG. 9, the real-time gait lines swing sharply, in which basically the center of pressures in each frame swings, a force is not applied on the fourth and fifth metatarsals, and shifted forward to the first, second, third, and fourth metatarsals, resulting in the first and second metatarsals bearing force excessively. The gait line which does not approach to the foot type and is not smooth, i.e. the gait line being tortuous, indicates that an origin of force of the tester' walking is swinging, which will cause uneven dynamic stress distribution. And the origin of the force swings on the hindfoot and directly reaches out the second and third metatarsals without passing through the arch, resulting in excessive force on the forefoot and unsteady walking, which is consistent with the test result of the plantar static pressure data.

(3) Preparation of the insole body: performing insole modeling by using an orthotics module database in an Easy CAD software, based on the plantar static pressure data; calculating a thickness of the insole by importing the plantar dynamic pressure data, and manufacturing the insole body by using 3D printing technology;

(4) Partition of the insole body: dividing the insole body 10 into the first toe region 11, the second toe region 12, the first metatarsal region 13, the second metatarsal region 14, the medial arch region 15, the heel region 16, the lateral arch region 17, the fifth metatarsal region 18, the fourth metatarsal region 19, and the third metatarsal region 20. It should be noted that the partitions of the insole body is based on the partitions of the bones of the sole of the human foot, for example, the first toe region 11 corresponds to the thumb of the foot, and the second toe region 12 corresponds to the remaining 4 toes of the foot.

(5) Preparation of the convex cushion structure: determining the shape of the convex cushion structure by using the test result of the dynamic pressure distributions. It can be seen from the test chart of the dynamic pressure distribution data that, the pressure on forefoot is too large and the heel is unsteady. In order to increase steadiness, the convex cushion structure is designed as a triangle. Due to the dynamic stress distributions are uneven, the convex cushion structure 30 is set as an asymmetric structure. Since the stresses at the positions of the second and third metatarsals are different, the heights of the convex cushion structure are not uniform and have a certain smooth transition. Since the gait line is abnormal, the gait cycle swings, and the gait line does not pass through the foot arch and directly reaches out the second and third metatarsals, the convex cushion structure 30 is arranged near the second metatarsal region 14 and the third metatarsal region 20. The trend of the gait line lies in that the force is not applied to the fourth and fifth metatarsals, and is shifted forward to the first, second, third, and fourth metatarsals, which results in the first and second metatarsals bearing force excessively, and thus an arc 31 of the convex cushion structure 30 is biased towards the direction of the medial arch region 15. The convex cushion structure 30 is made by integrating data through a computer and using 3D printing technology to print on the upper surface of the insole body.

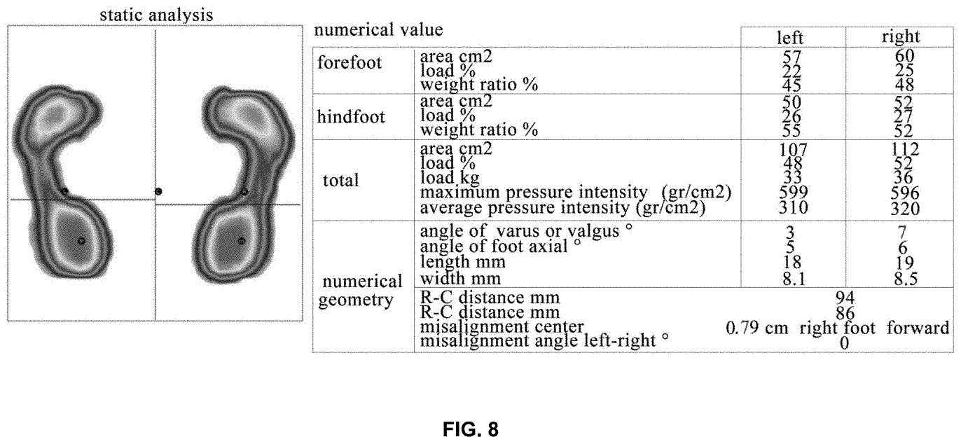

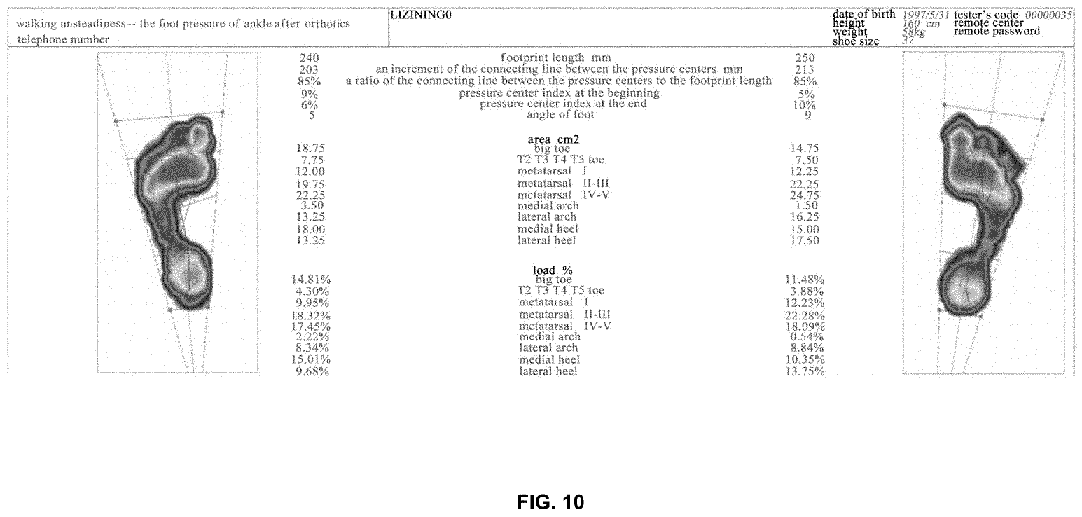

The tester wears the orthopedic insole prepared for 3 to 8 hours a day. And the data are collected one and a half months later, as shown in FIG. 8 and FIG. 10. FIG. 8 is a test chart of the tester's plantar static pressure data after orthotics, and FIG. 10 is a test chart of dynamic pressure distribution data after orthotics. FIG. 8 shows that the barefoot after orthotics is obviously closer to the theoretical value. FIG. 10 shows that the dynamic gait line does not have a lot of twists and turns, and it is much smoother and more similar to the foot type than it was more than a month ago. After more than a month of orthotics, the unsteadiness of the ankle joint was obviously cured.

Compared with the prior art, the manufacturing method of the convex cushion structure for walking unsteadiness according to the disclosure is designed by using the plantar static pressure data and the plantar dynamic pressure data, especially using three kinds of data, comprising the dynamic pressure distributions, the gait lines and the gait cycle, to perform analyses to obtain accurate data, which can maximize the advantages of conforming with personal sports biomechanics. The convex cushion structure is arranged on the upper surface of the insole body, which can effectively protect patients who walk unsteadily from uncomfortable walking, with simple preparation and low cost.

It should be pointed out that the above specific embodiments are only used to illustrate the disclosure and not to limit the scope of the disclosure. After reading the disclosure, various equivalent modifications according to the disclosure made by those skilled in the art all fall into the scope defined by the appended claims of the application.

What is claimed is:

1. A method of manufacturing a convex cushion structure for walking unsteadiness, the convex cushion structure being provided on an upper surface of an insole body, the method comprising:
collecting data by measuring, using a foot pressure plate, foot pressure data of a tester under two natural states of standing and walking, wherein the foot pressure data comprises plantar static pressure data and plantar dynamic pressure data, the plantar dynamic pressure data comprising dynamic pressure distributions, gait lines, and a gait cycle;
determining, via data analysis, whether pressure distributions of a left foot and a right foot of the tester are symmetrical, whether pressures on a forefoot and a hindfoot are too concentrated, whether a maximum force bearing point is moved forward, and whether there is toed-in or toed-out, according to the plantar static pressure data; and determining whether the gait lines are normal and a swing situation of the gait cycle according to the plantar dynamic pressure data, wherein, the dynamic pressure distributions are used to determine whether the pressure distributions of the left foot and the right foot are symmetrical, whether the pressures on the forefoot and the hindfoot are too concentrated, whether the maximum force bearing point is moved forward, and whether there is toed-in or toed-out, wherein the gait lines are used to determine whether there are situations of flatfoot, clawfoot, metatarsal pain, equinus heel pain, and unsteadiness of a center of gravity of the tester, and wherein the gait cycle is used to determine whether there is abnormal walking, and determine whether the center of gravity is unsteady in combination with the gait lines;
preparing, based on the plantar static pressure data, the insole body by performing insole modeling using an orthotics module database in an Easy CAD software, and calculating a thickness of the insole by importing the plantar dynamic pressure data, and manufacturing the insole body using 3D printing technology;
partitioning the insole body by dividing the insole body into a first toe region, a second toe region, a first metatarsal region, a second metatarsal region, a medial arch region, a heel region, a lateral arch region, a fifth metatarsal region, a fourth metatarsal region, and a third metatarsal region; and
preparing the convex cushion structure by determining a shape of the convex cushion structure by using a test result of the dynamic pressure distributions, and determining a specific location where the convex cushion structure is located on the upper surface of the insole body by using a test result of the gait lines and the gait cycle while determining a curve radian of the convex cushion structure by using the gait lines; and then printing out the convex cushion structure on the upper surface of the insole body by using the 3D printing technology.

2. The method of claim 1, wherein collecting data further comprises obtaining digital footprints using a 2D scanner to obtain a shape of each foot of the tester.

3. An orthopedic insole, comprising an insole body and a convex cushion structure provided on an upper surface of the insole body, the convex cushion structure being manufactured by the method of claim 1.

4. The orthopedic insole of claim 3, wherein collecting data further comprises obtaining digital footprints using a 2D scanner to obtain a shape of each foot of the tester.

5. The orthopedic insole of claim 4, wherein a bottom of the insole body is provided with a plurality of honeycomb structures.

6. The orthopedic insole of claim 5, wherein the plurality of the honeycomb structures penetrate the insole body.

7. The orthopedic insole of claim 6, wherein axial heights of the plurality of honeycomb structures are different.

8. The orthopedic insole of claim 6, wherein the honeycomb structure is formed as a hexagon.

9. The orthopedic insole of claim 5, wherein axial heights of the plurality of honeycomb structures are different.

10. The orthopedic insole of claim 5, wherein the honeycomb structure is formed as a hexagon.

11. The orthopedic insole of claim 10, wherein a distance between opposite sides of the hexagon is 2.5 mm.

12. The orthopedic insole of claim 11, wherein a distribution density of the honeycomb structures in the insole body is 8 pcs/cm².

13. The orthopedic insole of claim 3, wherein a bottom of the insole body is provided with a plurality of honeycomb structures.

14. The orthopedic insole of claim 13, wherein axial heights of the plurality of honeycomb structures are different.

15. The orthopedic insole of claim 13, wherein the plurality of the honeycomb structures penetrate the insole body.

16. The orthopedic insole of claim 15, wherein axial heights of the plurality of honeycomb structures are different.

17. The orthopedic insole of claim 15, wherein the honeycomb structure is formed as a hexagon.

18. The orthopedic insole of claim 13, wherein the honeycomb structure is formed as a hexagon.

19. The orthopedic insole of claim 18, wherein a distance between opposite sides of the hexagon is 2.5 mm.

20. The orthopedic insole of claim 19, wherein a distribution density of the honeycomb structures in the insole body is 8 pcs/cm².
